(19) United States
(12) Patent Application Publication
(10) Pub. No.: US 2008/0182903 A1
(43) Pub. Date: Jul. 31, 2008

(54) Formulation of a composition for treating various conditions by using lotion/cream including L-Arginine

(76) Inventor: Kaleem Ahmad, Plainfield, IL (US)

Correspondence Address: WILSON DANIEL SWAYZE, JR., 3804 CLEARWATER CT., PLANO, TX, 75025, US

(21) Appl. No.: 11/953,092

(22) Filed: Dec. 10, 2007

Related U.S. Application Data
(60) Provisional application No. 60/869,405, filed on Dec. 11, 2006.

(51) Int. Cl. A61K 31/198; A61P 1/00; A61P 1/04; A61P 15/00

(52) U.S. Cl. 514/565

(57) ABSTRACT

Disclosed is a composition and method, for various conditions, including body weight control, sexual dysfunction, stomach blotting, heartburn, body pain, and controlling cholesterol, arginine is used as active material. The composition is in the form of lotion/cream applicable to any part of the body and still functional. It does not need to be applied on problematic site.

### PRIORITY

The present invention claims priority based upon a provisional application filed on Dec. 11, 2006 with a Ser. No. 60/869,405.

### FIELD OF THE INVENTION

The present invention relates to a composition and a method for treating various conditions and more particularly for treating conditions including and not limited to excess body weight, sexual dysfunction, stomach blotting, heart burn, pain, toe nail fungus and controlling cholesterol.

### BACKGROUND

One problem that affects the human population is excess body weight which can lead to other problems such as high blood pressure or other forms of heart disease such as cholesterol. Another problem which affects a great percentage of the human population is sexual dysfunction. Stomach bloating affects a great number of people. Furthermore, the toenail fungus causes a significant amount of pain.

### SUMMARY

A method for treating a user with Arginine having a concentration substantially from 0.001 to 25%, in a carrier medium includes the step of applying the Arginine on any part of the body to treat the body for an abnormal condition.

The abnormal condition to be treated may be to control or lower body weight, and the abnormal condition may be to improve sexual dysfunction.

The abnormal condition to be treated may be to overcome stomach-blotting/stomach discomfort or gas problem, and the abnormal condition to be treated may be heartburn.

The Arginine may improve the digestive system, and the abnormal condition to be treated may be body pain.

The abnormal condition to be treated may be to control cholesterol, and the abnormal condition to be treated may be to control nail fungus/pain associated with in grown nail with fungus.

The abnormal condition to be treated may be to control one of the group consisting of heartburn, upset stomach, cholesterol or to control stomach blotting, and Arginine may be suspended or dissolved.

The Arginine may be in a cream or a lotion.

An agent to be administered to a user may include a free base Arginine with a concentration substantially from 0.001 to 25%; and the base may include Borax having a concentration substantially from 0.001 to 2.0%.

The base may include beeswax having a concentration substantially from 0.5 to 15%, and the base may include mineral oil having a concentration substantially from 1.0 to 15%.

The base may include cetyl alcohol having a concentration substantially from 0.01 to 5%, and the base may include Octyl Isononanoate having a concentration substantially from 0.1 to 7.0%.

The base includes one from the group consisting of Decyl Oleate having a concentrate substantially from 0.1 to 8.0%, Cetyl PEG/PPG1O/1 Dimethicone having a concentration substantially from 0.1 to 8.25%, Glyceryl Stearate (and) PEG-1OU Stearate having a concentration substantially from 0.1 to 10.5%, Cyclomethicone having a concentration substantially from 0.01 to 3.5%, Olive Oil having a concentration substantially from 0.01 to 5.5%, Almond Oil having a concentration substantially from 0.01 to 3.5%, Polyglycery1-4 Isostearate from 0.01 to 7.25%, Salt having a concentration substantially from 0.01 to LO %, Urea having a concentration substantially from 0.01 to 5.0%, Stearic acid having a concentration substantially from 0.01 to 3.0%, Triethanolamine from 0.001 to 1.5%, Vitamin E acetate having a concentration substantially from 0.01 to 1.0%, Zinc in its salt form, having a concentration substantially from 0.001 to 1.0%, Propylene Glycol having a concentration substantially from 0.1 to 8.25%, Methylparaben having a concentration substantially from 0.01 to 0.1O %, Propylparaben having a concentration substantially from 0.01 to 0.20% and remaining water to make 100%.

### DETAILED DESCRIPTION

The following description is the few of the conditions which can be treated by a composition of the present invention. The composition of the present invention may be in the form of a carrier including a lotion or cream that can be applied topically to any part of the body and is functional to provide benefits. The active component of the present invention is L-Arginine and the corresponding salts, and this active component is functional and helpful to improve or eliminate various conditions such as body weight, sexual dysfunction, stomach blotting, heartburn, pain, toe nail fungus and controlling cholesterol. The above-mentioned active component can be used even in a small quantity in a lotion or cream form and may be applied topically for these treatments (to any part of the body).

The present invention is based on the active component which may be in the form of a lotion or cream including L-Arginine in a small amount can reduce body weight when applied externally for a month or more on any part the body. A person applied the lotion including L-Arginine in a small amount once a day for one month on his skin. The user lost 9 pounds in weight and hip volume of the user was reduced by two inches.

In another aspect, the present invention provides a lotion or cream, when applied topically to any part of the body, helps to improve sexual dysfunction. A 55-year-old man used this lotion externally and applied once daily for one month, and his condition is significantly improved. Corresponding improvements may be achieved by females.

In another aspect, the present invention when applied has a lotion or cream and when applied topically to any part of the body, helps to overcome stomach-blotting problem. A woman applied lotion on the skin experiencing stomach discomfort, she felt better after applying lotion. The stomach discomfort was significantly reduced or eliminated.

In another aspect, the present invention can be used as a lotion or cream including L-Arginine in a small amount, when applied topically to any part of the body, helps to reduce or eliminate heartburn. A person having heart burn problem used this lotion for two weeks, and as a result, he felt better and his condition is improved. In conclusion, his digestive system was improved.

In another aspect, the present invention provides a lotion or cream including L-Arginine in a small amount, when applied topically to any part of the body, helps to reduce or eliminate pain associated with the shoulder, chest, arm, foot, knee and leg associated with overwork. A woman experienced pain in her foot after running on treadmill, and the pain continued for sometime. She applied this lotion including L-Arginine in a small amount on her foot, and she felt that the pain has been reduced or overcome. A person having arm pain applied lotion including L-Arginine in a small amount. As a result, he felt better. Other people having knee problem also used this lotion including L-Arginine in a small amount and their discomfort was reduced or eliminated. In another aspect, the present invention provides a lotion or cream including L-Arginine in a small amount, when applied topically to any part of the body, helps to control cholesterol. A person having slightly elevated cholesterol, used this lotion including L-Arginine in a small amount for two months, his condition is improved and his blood pressure is improved also.

In another aspect, the present invention provides a lotion or cream including L-Arginine in a small amount, when applied topically to toe nails once a day, reduced or eliminated nail fungus. A man having toe nail fungus applied this lotion or cream including L-Arginine in a small amount once a day, his nails and foot started clarifying, the color of the nails change back to normal and the in grown nail area of the nail was reduced or eliminated. It helped to minimize in-grown toenail pain.

A formulation of preferred embodiment of the present invention is shown as an example but is not limited to these ingredients, and other modifications are within the scope of the present invention.

In an example, L-Arginine or its salts may be used in a concentration from substantially 0.01 to 25% in as an agent, Borax may be used in a concentration substantially from 0.001 to 2.0%, Beeswax may be used in a concentration substantially from 0.5 to 15%, Mineral oil may be used in a concentration substantially from 1.0 to 15%, Cetyl Alcohol may be used in a concentration substantially from 0.01 to 5%, Octyl Isononanoate may be used in a concentration substantially from 0.1 to 7.0%, Decyl Oleate may be used in a concentration substantially from 0.1 to 8.0%, Cetyl PEG/PPG1O/1 Dimethicone may be used in a concentration from 0.1 to 8.25%, Glyceryl Stearate (and) PEG-100 Stearate may be used in a concentration substantially from 0.01 to 10.5%, Cyclomethicone may be used in a concentration substantially from 0.01 to 3.5%, Olive Oil may be used in a concentration substantially from 0.01 to 5.5%, Almond Oil may be used in a concentration substantially from 0.01 to 3.5%, Polyglycery1-4 Isostearate may be used in a concentration from 0.01 to 7.25%, Salt may be used in a concentration substantially from 0.01 to 1.0%, Urea may be used in a concentration substantially from 0.01 to 5.0%, Stearic acid may be used in a concentration substantially from 0.01 to 3.0%, Triethanolamine may be used in a concentration substantially from 0.001 to 1.5%, Vitamin E acetate may be used in a concentration substantially from 0.01 to 1.0%, Zinc which may be in its salt form, may be used in a concentration substantially from 0.01 to 1.0 Gb, Propylene Glycol may be used in a concentration substantially from 0.1 to 8.25% and remaining water may be used to make it 100%. In another aspect in which free base Arginine and its salts, from 0.001 to 25%, being suspended/dissolved in a carrier medium, or delivered in a capsule or tablet form or formulated in a base including gelling agents, thickeners, cream/lotion bases having a concentration substantially from 0.01 to 10.00%, propylene glycol having a concentration substantially from 0.01 to 10.00% and other agents to make it in a gel/cream/lotion form to control nail fungus.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

What is claimed is:

1. A method for treating a user with Arginine having a concentration substantially from 0.001 to 25%, in a carrier medium wherein the method includes the step of applying Arginine on any part of the body to treat the body for an abnormal condition.

2. A method for treating a user with Arginine having a concentration substantially from 0.001 to 25%, in a carrier medium as in claim 1, wherein the abnormal condition to be treated is to control or lower body weight.

3. A method for treating a user with Arginine having a concentration substantially from 0.001 to 25%, in a carrier medium as in claim 1, wherein the abnormal condition to be treated is to improve sexual dysfunction.

4. A method for treating a user with Arginine having a concentration substantially from 0.001 to 25%, in a carrier medium as in claim 1, wherein the abnormal condition to be treated is to overcome stomach-blotting/stomach discomfort or gas problem.

5. A method for treating a user with Arginine having a concentration substantially from 0.001 to 25%, in a carrier medium as in claim 1, wherein the abnormal condition to be treated is heartburn.

6. A method for treating a user with Arginine having a concentration substantially from 0.001 to 25%, in a carrier medium as in claim 1, wherein the Arginine improves the digestive system.

7. A method for treating a user with Arginine having a concentration substantially from 0.001 to 25%, in a carrier medium as in claim 1, wherein the abnormal condition to be treated is body pain.

8. A method for treating a user with Arginine having a concentration substantially from 0.001 to 25%, in a carrier medium as in claim 1, wherein the abnormal condition to be treated is to control cholesterol.

9. A method for treating a user with Arginine having a concentration substantially from 0.001 to 25%, in a carrier medium as in claim 1, wherein the abnormal condition to be treated is to control nail fungus/pain associated with in grown nail with fungus.

10. A method for treating a user with Arginine having a concentration substantially from 0.001 to 25%, in a carrier medium as in claim 1, wherein the abnormal condition to be treated is to control one of the group consisting of heartburn, upset stomach, cholesterol or to control stomach blotting.

11. A method for treating a user with Arginine having a concentration substantially from 0.001 to 25%, in a carrier medium as in claim 1, wherein the Arginine is suspended.

12. A method for treating a user with Arginine having a concentration substantially from 0.001 to 25%, in a carrier medium as in claim 1, wherein the Arginine is dissolved.

13. A method for treating a user with Arginine having a concentration substantially from 0.001 to 25%, in a carrier medium as in claim 1, wherein the Arginine is in a cream.

14. A method for treating a user with Arginine having a concentration substantially from 0.001 to 25%, in a carrier medium as in claim 1, wherein the Arginine is in a lotion.

15. An agent to be administered to a user, comprising:
a free base Arginine with a concentration substantially from 0.001 to 25%; and
wherein the base includes Borax having a concentration substantially from 0.001 to 2.0%.

16. An agent to be administered to a user as in claim 1, wherein the base includes beeswax having a concentration substantially from 0.5 to 15%.

17. An agent to be administered to a user as in claim 1, wherein the base includes mineral oil having a concentration substantially from 1.0 to 15%.

18. An agent to be administered to a user as in claim 1, wherein the base includes cetyl alcohol having a concentration substantially from 0.01 to 5%.

19. An agent to be administered to a user as in claim 1, wherein the base includes Octyl Isononanoate having a concentration substantially from 0.1 to 7.0%.

20. An agent to be administered to a user as in claim 1, wherein the base includes one from the group consisting of Decyl Oleate having a concentrate substantially from 0.1 to 8.0%, Cetyl PEG/PPG1O/1 Dimethicone having a concentration substantially from 0.1 to 8.25%, Glyceryl Stearate (and) PEG-1OU Stearate having a concentration substantially from 0.1 to 10.5%, Cyclomethicone having a concentration substantially from 0.01 to 3.5%, Olive Oil having a concentration substantially from 0.01 to 5.5%, Almond Oil having a concentration substantially from 0.01 to 3.5%, Polyglycery1-4 Isostearate from 0.01 to 7.25%, Salt having a concentration substantially from 0.01 to LO %, Urea having a concentration substantially from 0.01 to 5.0%, Stearic acid having a concentration substantially from 0.01 to 3.0%, Triethanolamine from 0.001 to 1.5%, Vitamin E acetate having a concentration substantially from 0.01 to 1.0%, Zinc in its salt form, having a concentration substantially from 0.001 to 1.0%, Propylene Glycol having a concentration substantially from 0.1 to 8.25%, Methylparaben having a concentration substantially from 0.01 to 0.10%, Propylparaben having a concentration substantially from 0.01 to 0.20% and remaining water to make 100%.
